(19) United States
(12) Patent Application Publication
(10) Pub. No.: US 2025/0235587 A1
(43) Pub. Date: Jul. 24, 2025

(54) SILVER FREE SURFACE COATING

(71) Applicant: BACTIGUARD AB, Stockholm (SE)

(72) Inventors: Javier SANCHEZ, Spånga (SE); Billy SÖDERVALL, Markaryd (SE)

(21) Appl. No.: 18/856,023

(22) PCT Filed: Apr. 5, 2023
(86) PCT No.: PCT/EP2023/058976

§ 371 Date: Oct. 10, 2024

(30) Foreign Application Priority Data
Apr. 12, 2022 (SE) 2250455-9

(51) Int. Cl. A61L 29/10; A61L 29/16; A61L 33/02

(52) U.S. Cl. CPC A61L 29/106; A61L 29/16; A61L 33/022; A61L 2300/102; A61L 2300/406; A61L 2300/42; A61L 2420/02

(57) ABSTRACT

There is provided an object, wherein there are particles on the surface of the object, wherein the amount of particles is in the interval 0.041-6 μg/cm², wherein the particles comprise palladium in an amount corresponding to 0.02-2 μg/cm², neodymium in an amount corresponding to 0.001-2 μg/cm², and gold in an amount corresponding to 0.02-2 μg/cm², and wherein the particles comprise less than 30 ppm silver, which is considered as silver free. The coating is both biocompatible and antimicrobial and does not suffer from disadvantages associated with silver since the coating is silver free. The coating shows improved action against thrombosis.

### FIELD OF THE INVENTION

The present invention relates to a surface coating comprising particles of palladium, gold and neodymium, which coating is silver free.

### BACKGROUND

Surfaces with antimicrobial and biocompatible properties are important within many applications. Examples of surfaces where such properties are of importance include surfaces intended to be in contact with a human or animal body including contact with the skin as well as body cavities and inside a body. Medical equipment, which is intended to be in contact with human or animal blood, should preferably have properties so that formation of blood clots and thrombosis is avoided.

U.S. Pat. No. 6,224,983 discloses an article with an adhesive, antimicrobial and biocompatible coating comprising a layer of silver stabilised by exposure to one or more salts of one or more metals selected from the group consisting of platinum, palladium, rhodium, iridium, ruthenium and osmium. The thickness of the silver layer is in the range 2-2000 Å (Å, Ångström, Angstrom, 10⁻¹⁰ m) and further disclosed ranges are 2-350 Å and 2-50 Å. There are also examples of a thickness of the silver layer of 50 Å, 350 Å, 500 Å, and 1200 Å. The substrate may be latex, polystyrene, polyester, polyvinylchloride, polyurethane, ABS polymers, polycarbonate, polyamide, polytetrafluoroethylene, polyimide or synthetic rubber.

WO2007/117191, WO2007/117213 and WO2007/117214 disclose a substrate having an electron donating surface, characterized in that there are metal particles on said surface, the metal particles comprise palladium and at least one metal selected from the group consisting of gold, ruthenium, rhodium, osmium, iridium, and platinum and wherein the amount of the metal particles is from about 0.001 to about 8 μg/cm².

Kris N.J. Stevens et al. in Biomaterials 30 (2009) 3682-3690 discloses that surface coatings for medical devices can be made antimicrobial through introduction of silver nanoparticles. By virtue of their extremely large surface-to-volume ratio, the silver particles serve as a depot for sustained release of silver ions, despite the fact that silver is not readily oxidized. The study specifically addresses the question what the impact of silver nanoparticles (exposed at the coating's surface) and/or the release of silver ions would be on coagulation of contacting blood. It is concluded that the observed activation of blood platelets can be best explained through a collision mechanism. The results suggest that platelets that collide with silver, exposed at the surface, become activated without adhering to the surface. These new results point, rather unexpectedly, at a double effect of the silver nanoparticles in the coating: a strong antimicrobial effect occurs simultaneously with acceleration of the coagulation of contacting blood.

WO 2007/142579 discloses a polymer matrix, characterized in that it comprises a. an electron donating constituent and b. metal particles comprising at least one metal selected from the group consisting of palladium, gold, ruthenium, rhodium, osmium, iridium, and platinum.

WO 2019/206950 discloses a method for decreasing leakage of matter from an object to a surrounding, said object being coated with a coating at least partially applied on the object, said coating comprising an at least partially covering layer comprising silver, said object optionally comprising area(s) without said layer, said coating comprising metal particles applied on the layer and optionally on areas without said layer, said metal particles comprising palladium and at least one metal selected from the group consisting of gold, ruthenium, rhodium, osmium, iridium, niobium, neodymium and platinum and wherein the amount of the metal particles is in the interval 0.01-8 μg/cm². It is disclosed that blood clotting can be reduced if the surface is exposed to blood from a human or animal, when the metal particles comprise palladium and neodymium. Silver is always present in the coating.

Although the amount of silver, which is released from coatings such as the coatings, described in WO 2019/206950 is minimal, the silver may anyway have disadvantages in some cases. When applied to certain materials the silver may cause discoloration in spite of being applied in relatively low amounts according to the state of the art such as in WO 2019/206950. Further, the application of silver requires some steps during the manufacture. The silver is typically applied form baths, which have to be replaced regularly.

Even though WO 2019/206950 shows that blood clotting can be reduced to a significant and useful extent if the surface is exposed to blood from a human or animal, there is still room for further improving the action against thrombosis.

Another problem in the state of the art is how to provide a silver-free coating, which is still antimicrobial and biocompatible.

### SUMMARY

One object of the present invention is to obviate at least some of the disadvantages in the prior art and provide an improved surface coating.

In a first aspect there is provided an object, wherein there are particles on the surface of the object, wherein the amount of particles is in the interval 0.041-6 μg/cm², wherein the particles comprise palladium in an amount corresponding to 0.02-2 μg/cm², neodymium in an amount corresponding to 0.001-2 μg/cm², and gold in an amount corresponding to 0.02-2 μg/cm², and wherein the particles comprise less than 30 ppm silver.

There is further provided use of such an object for prevention of thrombosis. There is also provided use of the coating for preventing of thrombosis.

Further embodiments of the present invention are defined in the appended dependent claims, which are explicitly incorporated herein.

One advantage is that the anti thrombosis effect is improved.

Further, the surface has an improved antibacterial action compared to a similar surface including silver.

The manufacture of the surface is simpler since no silver has to be deposited during the manufacture.

### BRIEF DESCRIPTION OF THE DRAWINGS

Aspects and embodiments will be described with reference to the following drawings in which:

FIG. 1 shows an example of a surface of an object according to the invention. The picture is from example 2, i.e. the coating on nitinol tubes. The particles comprise palladium, neodymium, and gold.

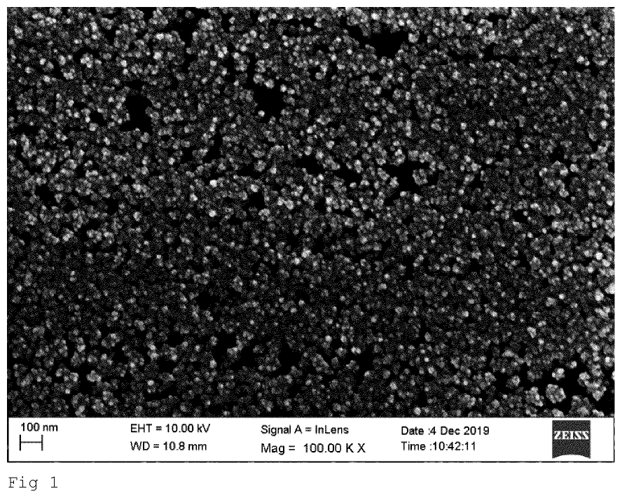

FIG. 2 shows another example of a surface of an object according to the invention at a higher magnification. This picture is from example 1, i.e. the coating on the alloy Ti-6Al-4V.

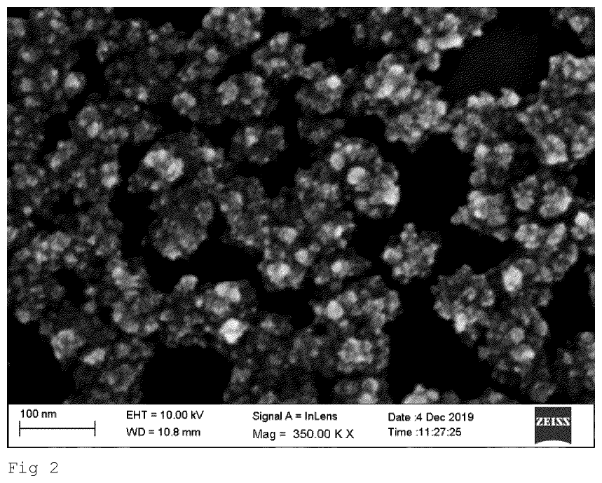

### DETAILED DESCRIPTION

Before the invention is disclosed and described in detail, it is to be understood that this invention is not limited to particular configurations, process steps and materials disclosed herein as such configurations, process steps and materials may vary somewhat.

It must be noted that, as used in this specification and the appended claims, the singular forms “a”, “an” and “the” include plural referents unless the context clearly dictates otherwise.

The following terms are used throughout the description and the claims.

“Amount” of particles or other material on a surface is herein often given as μg/cm². This is a suitable way of expressing the amount since the applied layer is very thin. For calculating the amount, the coated area of the object is measured and the amount per the coated area is calculated.

“Antimicrobial” as used herein is the property of suppressing or eliminating microbial growth. Microbial growth includes but is not limited to bacterial growth.

“Biocompatible” as used herein is the ability of a material to perform with an appropriate host response in a specific application.

“Object” as used herein is the substrate, which is treated and at least partially surface coated according to the present invention.

“Silver free” as used herein means that the coating is practically free of silver, which is interpreted so that the particles in the coating comprise less than 30 ppm silver.

According to the present invention, a surface coating is applied on an object to give it desired properties. The particles on the surface of the object can be viewed as a surface coating. The object can be made of a wide range of materials.

In the first aspect there is provided an object, wherein there are particles on the surface of the object, wherein the amount of particles is in the interval 0.041-6 μg/cm², wherein the particles comprise palladium in an amount corresponding to 0.02-2 μg/cm², neodymium in an amount corresponding to 0.001-2 μg/cm², and gold in an amount corresponding to 0.02-2 μg/cm², and wherein the particles comprise less than 30 ppm silver.

The object is at least partially coated with metal particles on its surface. The metal particles are deposited on the surface of the object. The object is at least partially coated with particles so that there may be areas on the object without particles. In one embodiment, the entire object is coated with particles. An area, which is not coated with particles, is characterized in that the distance to the nearest particle on the surface is more than 1 mm. For a coating the particles on the surface are so abundant that the distance between the particles is much less than 1 mm, the distance between particles on the surface is several order of magnitude less than 1 mm. Areas without the coating can then by identified as having no particles. In order to determine an exact boundary where the coating ends, the criterion of 1 mm to the nearest particle can be used. This criterion makes it easy to distinguish coated and non-coated areas since the distance between particles is very much lower than 1 mm in coated areas and since there are virtually no particles in non-coated areas. Non-coated areas have typically not been dipped or have been masked in some way.

The particles always comprise palladium, neodymium, and gold. The amounts of the different metals in the particles are calculated based on the weight of metal per area of the object. The total amount of the particles is also calculated based on the weight of the particles per area of the object. The total amount of the particles may comprise additional additives in addition to the three compulsory metals. It should however be noted that the amount of impurities in general should be kept as low as possible since the coating is intended for medical products and the effect of additional impurities may be difficult to determine.

The surface coating is free of silver, which is interpreted so that the particles in the coating comprise less than 30 ppm silver. Any avoidable silver should be added neither to the particles nor to the surface of the object. Even though no silver is added to the coating, the other metals in the surface coating may comprise small amounts of various impurities including silver. Thus, when the other metals are added it cannot be ruled out that there are some small amounts of silver as an impurity. Very small amounts of silver may be unavoidable because of impurities in the metals. For instance, if gold is added there may be a small amount of silver as an impurity in the gold. Thus, a very small amount of silver may be difficult to avoid even if silver is not deliberately added. The amount of silver should be kept as low as practically possible. Since the amount of silver in commercially available high purity grades of the metals palladium, gold and neodymium is in the order of 10-30 ppm it may be difficult to reach lower amounts of silver in a commercial scale. It is thus motivated to refer to the surface as silver-free when the amount of silver in the particles is less than 30 ppm. By using higher quality grades of metals, it is possible to reach a silver amount in the particles of less than 20 ppm and even less than 10 ppm. It may even be possible to reach a silver amount of less than 5 ppm.

The object onto which the particles are deposited should also not comprise silver, at least it should comprise as little silver as possible, otherwise the idea of a silver free coating would not be effective. The outermost 10 μm of the object comprises less than 30 ppm silver. The distance from the surface is measured in a direction perpendicular to the surface and 10 μm into the object. In this part of the object the amount of silver is calculated by weight. The amount of silver in the outermost 10 μm is measured or calculated before application of the particles.

In one embodiment the object before application of the particles does not comprise more than 30 ppm silver. This embodiment takes into account the entire uncoated object, which should not comprise more than 30 ppm silver by weight.

The object onto which the particles are deposited does not comprise a coating comprising silver.

In one embodiment, the entire object including the particles, i.e. including the coating comprises less than 30 ppm silver, preferably less than 20 ppm silver, more preferably less than 10 ppm silver. In one embodiment, the object including the particles on the surface of the object comprises less than 30 ppm silver.

The amount of silver is calculated by weight in ppm (parts per million 10⁻⁶). 1 ppm as used herein corresponds to 0.0001 wt %.

In one embodiment the particles comprise at least one metal selected from ruthenium and rhodium. Rhodium has the effect of further decreasing leakage of matter from the object. Examples of matter, which may leak, is allergens and ions originating from the coated object. An addition of rhodium in the metal particles can increase this effect. The effect of decreasing leakage of matter is a chemical effect and no physical barrier. This is evident since the distance between the particles is so that the coating cannot form a physical barrier. Rhodium gives very good results to prevent leakage of matter. In one embodiment, the amount of rhodium in the particles corresponds to 0.05-2 μg/cm². For embodiments with a higher amount of rhodium in the particles, the amount of particles on the object can be higher than 6 μg/cm². In one embodiment where the particles comprise rhodium, the amount of particles is in the interval 0.091-8 μg/cm².

Regarding ruthenium, an effect of ruthenium is to decrease leakage of matter over time. When adding ruthenium the leakage of matter is decreased over time. In one embodiment, 0.05-2 μg/cm² of ruthenium is added. For embodiments with a higher amount of rhodium and/or ruthenium in the particles, the amount of particles on the object can be higher than 6 μg/cm². In one embodiment where the particles comprise ruthenium, the amount of particles is in the interval 0.091-8 μg/cm². In one embodiment where the particles comprise ruthenium and rhodium, the amount of particles is in the interval 0.141-8.2 μg/cm².

In one embodiment, the object comprises at least one metal. Since the coating is to be silver-free, the object to be coated should also comprise as little silver as possible and thus the metal in the object cannot be silver. As discussed above a silver amount of maximum 30 ppm silver in the object is a suitable limit.

In one embodiment, the at least one metal is selected from the group consisting of iron, titanium, cobalt, nickel, chromium, and mixtures thereof.

In one embodiment, the at least one metal is selected from the group consisting of steel, stainless steel, and nitinol.

In one embodiment the at least one metal is selected from the group consisting of medical grade titanium, medical grade stainless steel, and medical grade nitinol. The wording medical grade indicates that the metal or alloy is intended for medical use such as for implants or for being in contact with a human or animal body. Examples of medical grade stainless steel include but are not limited to SAE 316, SAE 440, SAE 420, and 316L produced according to ASTM F138/F139.

In one embodiment, the object comprises at least one ceramic material. A ceramic material is a material made by firing an inorganic, non-metallic material.

In one embodiment, the object comprises at least one selected from the group consisting of silicon nitride, and zirconium dioxide.

In one embodiment, the object comprises at least one selected from the group consisting of apatite, and hydroxyapatite.

In one embodiment, the object comprises at least one polymer.

In one embodiment, the object comprises at least one polymer composite. A polymer composite comprises short or continuous fibres bound together by a matrix of organic polymers. In a polymer composite, a polymer is combined with various continuous and non-contiguous reinforcements/fibres, typically added to the polymer to improve the material performance.

In one embodiment the polymer is selected from the group consisting of latex, vinyl, polymers comprising vinyl groups, polyurethane urea, silicone, polyvinylchloride, polypropylene, styrene, polyurethane, polyester, copolymerisates of ethylene vinyl acetate, polytetrafluoroethylene (PTFE), polyether ether ketone (PEEK), polystyrene, polycarbonate, polyethylene, polyacrylate, polymethacrylate, acrylonitrile butadiene styrene (ABS), polyamide, polyimide, and mixtures thereof. These polymers can be a part of a polymer composite or form an object.

In one embodiment, the object comprises at least one textile material. A textile is a material made of interlacing fibres.

It must be noted that the particles are essentially homogenously composed, i.e. they have essentially the same composition of metals throughout the particle.

The amount of particles on the surface is 0.041-6 μg/cm², however if additional metals in addition to the three compulsory metals are used, then the amount of the particles can be a bit higher such as up to 8 μg/cm² or up to 10 μg/cm². In one embodiment, the amount of the particles is in the interval 0.05-2 μg/cm². The amounts refer to the total weight of the particles in relation to the coated part of the object.

In one embodiment, the particles are separated particles, not in contact with each other, i.e. sparsely distributed particles, which are not in contact with each other. In an alternative embodiment, parts of the particles are in contact with each other to form agglomerates of particles. In such agglomerates, a number of particles are in contact to form an agglomerate. Nevertheless, the surface of the object is still accessible to an aqueous solution so that the coating with particles is percolated and permeable to aqueous solutions.

In one embodiment, the particles have a size in the interval 10-500 nm. The particle size is measured according to ISO 19749:2021 by scanning electron microscopy. A skilled person is aware that there are other methods of characterizing a surface such as an electron probe microanalyzer (EPMA) with WDX detectors and calculating a particle size from simulations of the measured data. However, the particle sizes as defined in the description and in the claims are as defined in ISO 19749:2021. In one embodiment, the particles have a size in the interval 10-200 nm. In one embodiment, the particles have a size in the interval 10-60 nm. A person skilled in the art realises that the particle size can be in different intervals from about 10 to about 500 nm. Examples of such intervals include but are not limited to 10-400 nm, 10-300 nm, 10-100 nm, 10-80 nm, 10-70 nm, 10-60 nm, 15-150 nm, 15-100 nm, 15-60 nm, 20-80 nm, 20-60 nm, 25-50 nm, and 30-40 nm.

Examples of objects comprising a substrate according to the present invention include but are not limited to medical devices, medical instruments, and medical disposable articles. In one embodiment, the object is selected from medical devices, medical instruments, and medical disposable articles. A medical device is a device, intended for medical use. A medical instrument is an instrument, intended for medical use. A medical disposable article is a disposable article, intended for medical use.

In one embodiment, the object is an object intended to be in contact with human or animal blood. This use is particularly beneficial since the formation of blood clots is reduced compared to other materials. The risk for thrombosis is reduced. Objects intended to be inside a human or animal body or in contact with human or animal blood are suitable to coat according to the present invention. As an example, objects in devices intended to be in contact with human or animal blood are coated. Such objects include machines and devices where human or animal blood is processed and returned to a human or animal. For such procedures, the risk of thrombosis is reduced.

In one embodiment the object is at least one selected from the group consisting of a catheter, a central venous catheter, a peripheral venous catheter, a urinary catheter, a Foley catheter, an intermittent catheter, an implant, a dental implant, a dental abutment, a dental aligner, a dental prosthesis device, a bone replacing implant, an orthopaedic implant, a tissue replacing implant, a stent, a biliary stent, a tracheal stent, a peripheral stent, a glove, a pacemaker, a rupture net, a surgical instrument, a blood bag, an artificial heart valve, a vascular port, a haemodialysis equipment, a peritoneal dialysis equipment, a plasmapheresis device, an ecmo machine, a cardiopulmonary bypass device, an inhalation drug delivery device, a vascular graft, an arterial vascular graft, a venous vascular graft, a cardiac assist device, a wound dressing, an ECG electrode, an orthopaedic device, an intraocular lens, a suture, a needle, a staple, a mesh, a drug delivery device, an endotracheal tube, a shunt, a drain, a suction device, a hearing aid device, an urethral medical device, and an artificial blood vessel.

According to the invention, catheters can be coated. Examples of catheters include but are not limited to central venous catheters, peripheral venous catheters, urinary catheters, Foley catheters, and intermittent catheters.

Objects intended for dental use can be coated according to the invention. Examples of such objects include but are not limited to dental implants, dental abutments, dental aligners, and dental prosthesis devices.

According to the invention, implants can be coated. Implants for both dental applications as well as all other applications can be coated. Examples of implants include but are not limited to bone replacing implants, orthopaedic implants, and tissue replacing implants.

Stents can be coated according to the invention. Examples of stents include but are not limited to biliary stents, tracheal stents, and peripheral stents.

Objects intended to be in contact with human or animal blood during at least a part of their intended use can be coated according to the invention. Such objects include parts of devices and equipment, which are intended to be exposed to blood. Examples of such objects include but are not limited to haemodialysis equipment, peritoneal dialysis equipment, plasmapheresis devices, ecmo (Extracorporeal membrane oxygenation) machines, and cardiopulmonary bypass devices. For such objects, it is understood that the parts intended to come into contact with the blood are suitable to be coated.

Objects intended to be in contact with human or animal blood are suitable to coat and further examples include but are not limited to vascular grafts, arterial vascular grafts, venous vascular grafts, artificial blood vessels, artificial heart valves, blood bags, and vascular ports.

Also further objects are suitable to coat according to the invention and such objects include but are not limited to gloves, cardiac assist devices, pacemakers, rupture nets, surgical instruments, inhalation drug delivery devices, wound dressings, ECG electrodes, orthopaedic devices, intraocular lenses, sutures, needles, staples, meshes, drug delivery devices, endotracheal tubes, shunts, drains, suction devices, hearing aid devices, and urethral medical devices.

The invention is an improvement/derivative of the materials defined for instance in U.S. Pat. No. 5,320,908. Differences include but are not limited to that the layer of silver in U.S. Pat. No. 5,320,908 is excluded. In the present invention there are instead particles comprising neodymium, gold and palladium. The metals in the particles are also different compared to the metals in the layer in U.S. Pat. No. 5,320,908. In summary, it is an improvement of the materials described in U.S. Pat. No. 5,320,908.

The applied amount of the particles as well as the amount of the metals in the particles is expressed in μg/cm². This is calculated as the weight of the particles in relation to the coated area. It is the weight of the metal in relation to the coated area.

Now there is described one embodiment of the present invention for preparation of the coating. In one embodiment, the method includes the following steps:
1. rinsing (optional)
2. activation
3. rinsing (optional)
4. deposition of particles
5. rinsing (optional)
6. drying (optional)

Although the initial rinsing is optional, it is recommended.

The activation is made in an aqueous solution of a stannous salt containing 0.0005 to 30 g/l of stannous ions. The pH is 1 to 4 and adjusted by hydrochloric and/or sulphuric acid. The treatment time is 2-60 minutes at room temperature. After the pre-treatment, the surface is rinsed in demineralised water, but not dried.

In addition to the above activation, a treatment can be carried out before the activation in the stannous salt. Such an additional treatment is in one embodiment selected from the group consisting of treatment in alkali solution followed by neutralization in an acid solution, treatment in an NaOH solution followed by neutralization in HCl, treatment in an alkali solution upon heating to less than 90° C., treatment in an alcohol, and treatment in isopropanol.

Some polymeric objects are known to be difficult to coat in general such as for instance polytetrafluoroethylene (PTFE). For such difficult objects comprising for instance polytetrafluoroethylene (PTFE), polyether ether ketone (PEEK), polypropylene, and hydroxyapatite, an alternative pre-treatment can be used to improve the adhesion to the object. In one embodiment, a pre-treatment is performed before the coating. A plasticizer based on an aliphatic polyisocyanate is dissolved in a solvent. Suitable solvents include but are not limited to n-butyl acetate, isopropanol, and xylene. The dissolved plasticizer is applied to the object to be coated and then dried. The concentration of plasticizer is adapted so that the dried layer of plasticizer is only a few molecules thick in one embodiment. For such a thin coating, there are no essential changes in most of the physical properties of the object. When the surface has been cured, the coating can proceed. By using this pre-treatment good adhesion is obtained for difficult objects comprising polytetrafluoroethylene (PTFE), polyether ether ketone (PEEK) with carbon composite filler, nonwoven materials based on polypropylene, and hydroxyapatite. After the pre-treatment, the object is rinsed in demineralized water in one embodiment.

In one embodiment, colloidal suspensions of metals are used to obtain particles on the surface. The particles comprise a mixture of metals so that the desired composition is reached, i.e. all of the particles comprise the desired composition of metals. The particles are deposited from a suspension of the desired particles. The composition of the particles in the suspension is adjusted according to the desired amounts of the metals to be in the particles. The object is dipped in the suspension of particles for a period from about a few seconds to about a few minutes or longer.

The substrate is treated with the suspension for a period from about a few seconds to about a few minutes or longer. After the treatment, the substrate is rinsed in a solvent or water such as demineralised water and left to dry in room temperature.

The presence of palladium together with the activation with stannous ions gives an autocatalytic action so that the metal salts of palladium, neodymium, gold and other optional metals are reduced to elemental metal in particles.

Other features of the invention and their associated advantages will be evident to a person skilled in the art upon reading the description and the examples. It is understood that the disclosed embodiments can be freely combined with all other embodiments as long as it is not clearly contradictory.

It is to be understood that this invention is not limited to the particular embodiments shown here. The following examples are provided for illustrative purposes.

In the following, each of the described methods, apparatuses, examples and aspects, which do not fully correspond to the invention as defined in the claims is thus not according to the invention and is, as well as the whole following description, present for illustration purposes only or to highlight specific aspects or features of the claims

### EXAMPLES

### Example 1

A substrate of the common implant material Ti-6Al-4V was coated. This is an alpha beta titanium alloy. First the material was cleaned and rinsed in RO/DI (reverse osmosis demineralized) water. After the cleaning, the material was treated in a bath containing Sn²⁺ ions as a sensitizing step. The bath comprised a solution of aqueous stannous chloride. After rinsing in RO/DI water this was followed by immersing in a bath comprising a mixture of ions of Pd, Au and Nd. The metal ions were present as chlorides. The metals formed particles and deposited on the object due to the autocatalytic effect of palladium. The amount of the metals deposited on the titanium alloy surface was Pd 0.6 μg/cm², Au 0.2 μg/cm² and Nd 0.04 μg/cm².

A control coating with silver was also made where Pd 0.5 μg/cm², Au 0.4 μg/cm², Nd 0.06 μg/cm², and Ag 1.3 μg/cm² was deposited.

An uncoated control sample was also made, where no metals were deposited.

Thrombin-antithrombin complex (TAT) is a complex of thrombin and antithrombin and was used as a marker of net activation of blood coagulation. TAT values were measured after contact with human blood.

A comparison of the measured TAT values that indicates the tendency of thrombosis showed following results obtained from a TAT complex analysis.

| | Coating with Pd, | Coating with Pd, |
|---|---|---|
| Control | Au, Ag, and Nd | Au, and Nd |
| 2290 ng/ml | 280 ng/ml | 213 ng/ml |

As can be seen the results show a further decrease in TAT value when Ag is excluded.

### Example 2

A coating process similar to the process in example 1 was performed for tubes of the alloy Nitinol.

An uncoated control sample was made as well where no metal was deposited. A control sample including silver was made as well.

The resulting amounts were Pd 1.1 μg/cm², Au 0.6 μg/cm², and Nd 0.06 μg/cm². For the silver control sample the amounts were Pd 0.7 μg/cm², Au 0.6 μg/cm², Nd 0.04 μg/cm², and Ag 1.1 μg/cm².

The TAT values were measured and were the following:

| | Coating with Pd, | Coating with Pd, |
|---|---|---|
| Control | Au, Ag, and Nd | Au, and Nd |
| 852 ng/ml | 101 ng/ml | 52 ng/ml |

As can be seen the results show a clear decrease in TAT value when Ag is excluded.

### Example 3

A coating process similar to the process in example 1 was performed on silicone of medical grade.

A control sample was made as well, where no metals were deposited. Further, a control sample also including 1.5 μg/cm² silver was made.

Corresponding coating on silicone showed following values:

| | Coating with Pd, | Coating with Pd, |
|---|---|---|
| Control | Au, Ag, and Nd | Au, and Nd |
| 46 ng/ml | 23 ng/ml | 12 ng/ml |

As can be seen the results show a clear decrease in TAT value when Ag is excluded also for silicone.

### Example 4

A coating process similar to the process in example 1 was performed on polyurethane haemodialysis catheters.

A control sample was made as well, where no metals were deposited. A control sample including silver was also made.

Coating on polyurethane haemodialysis catheters showed the following amounts: Pd 0.6 μg/cm², Au 0.4 μg/cm², Nd 0.05 μg/cm². The control sample with silver had the following amounts: Pd 0.8 μg/cm², Au 0.3 μg/cm², Nd 0.05 μg/cm², and Ag 1.0 μg/cm².

A control sample was made as well, where no metals were deposited.

| | Coating with Pd, | Coating with Pd, |
|---|---|---|
| Control | Au, Ag, and Nd | Au, and Nd |
| 6425 ng/ml | 95 ng/ml | 54 ng/ml |

Also for this case, an improvement is seen when excluding silver.

### Antibacterial Properties

In all tests both with and without Ag, the Ahearn test was used for measuring the percentage of reduction of bacterial growth and the result was within 95-100%. However, an additional test measuring the viability showed no reactions with Ag but reaction up to 50% on the coatings with Nd. To ensure that no killing of bacteria had happened the test Zone of Inhibition was done and there were no reactions.

The conclusion is that the Nd coating without Ag has a higher reduction of thrombosis and it seems likely that the reduction of bacterial growth also is stronger.

1. An object, wherein there are particles on the surface of the object, wherein the amount of particles is in the interval 0.041-6 μg/cm², wherein the particles comprise palladium in an amount corresponding to 0.02-2 μg/cm², neodymium in an amount corresponding to 0.001-2 μg/cm², and gold in an amount corresponding to 0.02-2 μg/cm², and wherein the particles comprise less than 30 ppm silver, and wherein the outermost 10 μm of the object comprises less than 30 ppm silver.

2. The object according to claim 1, wherein the object including the particles on the surface of the object comprises less than 30 ppm silver.

3. The object according to claim 1, wherein the particles comprise at least one metal selected from the group consisting of ruthenium and rhodium.

4. The object according to claim 1, wherein the particles comprise less than 20 ppm silver.

5. The object according to claim 1, wherein the particles comprise less than 10 ppm silver.

6. The object according to claim 1, wherein the object is selected from the group consisting of medical devices, medical instruments, and medical disposable articles.

7. The object according to claim 1, wherein the object is any object intended to be in contact with human or animal blood.

8. The object according to claim 1, wherein the object comprises at least one metal.

9. The object according to claim 8, wherein the at least one metal is selected from the group consisting of iron, titanium, cobalt, nickel, chromium, and mixtures thereof.

10. The object according to claim 8, wherein the at least one metal is selected from the group consisting of steel, stainless steel, and nitinol.

11. The object according to claim 8, wherein the at least one metal is selected from the group consisting of medical grade titanium, medical grade stainless steel, and medical grade nitinol.

12. The object according to claim 1, wherein the object comprises at least one ceramic material.

13. The object according to claim 1, wherein the object comprises at least one selected from the group consisting of silicon nitride and zirconium dioxide.

14. The object according to claim 1, wherein the object comprises at least one selected from the group consisting of apatite and hydroxyapatite.

15. The object according to claim 1, wherein the object comprises at least one polymer.

16. The object according to claim 1, wherein the object comprises at least one polymer composite.

17. The object according to claim 15, wherein the polymer is selected from the group consisting of latex, vinyl, polymers comprising vinyl groups, polyurethane urea, silicone, polyvinylchloride, polypropylene, styrene, polyurethane, polyester, copolymerisates of ethylene vinyl acetate, polytetrafluoroethylene (PTFE), polyether ether ketone (PEEK), polystyrene, polycarbonate, polyethylene, polyacrylate, polymethacrylate, acrylonitrile butadiene styrene (ABS), polyamide, polyimide, and mixtures thereof.

18. The object according to claim 1, wherein the object comprises at least one textile material.

19. The object according to claim 1, wherein the amount of the particles is in the interval 0.05-2 μg/cm².

20. The object according to claim 1, wherein the particles are separated particles, not in contact with each other.

21. The object according to claim 1, wherein at least a part of the particles are in contact with other particles to form agglomerates of particles.

22. The object according to claim 1 wherein the neodymium is present in an amount corresponding to 0.001-0.15 μg/cm².

23. The object according to claim 1, wherein the particles have a size in the interval 10-500 nm as measured according to ISO 19749:2021.

24. The object according to claim 1, wherein the particles have a size in the interval 20-100 nm as measured according to ISO 19749:2021.

25. The object according to claim 1, wherein the object is at least one selected from the group consisting of a catheter, a central venous catheter, a peripheral venous catheter, a urinary catheter, a Foley catheter, an intermittent catheter, an implant, a dental implant, a dental abutment, a dental aligner, a dental prosthesis device, a bone replacing implant, an orthopaedic implant, a tissue replacing implant, a stent, a biliary stent, a tracheal stent, a peripheral stent, a glove, a pacemaker, a rupture net, a surgical instrument, a blood bag, an artificial heart valve, a vascular port, a haemodialysis equipment, a peritoneal dialysis equipment, a plasmapheresis device, an ecmo machine, a cardiopulmonary bypass device, an inhalation drug delivery device, a vascular graft, an arterial vascular graft, a venous vascular graft, a cardiac assist device, a wound dressing, an ECG electrode, an orthopaedic device, an intraocular lens, a suture, a needle, a staple, a mesh, a drug delivery device, an endotracheal tube, a shunt, a drain, a suction device, a hearing aid device, an urethral medical device, and an artificial blood vessel.

26. Use of an object for preventing thrombosis, wherein there are particles on the surface of the object, wherein the amount of particles is in the interval 0.041-6 μg/cm², wherein the particles comprise palladium in an amount corresponding to 0.02-2 μg/cm², neodymium in an amount corresponding to 0.001-2 μg/cm², and gold in an amount corresponding to 0.02-2 μg/cm², and
wherein the particles comprise less than 30 ppm silver and wherein the outermost 10 μm of the object comprises less than 30 ppm silver.
